(19) United States
(12) Patent Application Publication
(10) Pub. No.: US 2026/0273493 A1
(43) Pub. Date: Sep. 17, 2026

(54) METHOD AND DEVICE FOR AUTOMATION OF CHEMICAL SYNTHESIS

(71) Applicants: Sourav CHATTERJEE, Bath (GB); Sooyeon MOON, Zürich (CH)

(72) Inventors: Sourav CHATTERJEE, Bath (GB); Sooyeon MOON, Zürich (CH)

(21) Appl. No.: 19/510,608

(22) PCT Filed: Jul. 16, 2024
(86) PCT No.: PCT/EP2024/070095

§ 371 Date: Feb. 11, 2026

(30) Foreign Application Priority Data
Jul. 18, 2023 (CH) CH000776/2023

(51) Int. Cl. B01J 19/00; G16C 20/10; G16C 20/90

(52) U.S. Cl. CPC B01J 19/004; G16C 20/10; G16C 20/90

(57) ABSTRACT

The present disclosure relates to a chemical synthesizer (1) and a method for performing a chemical synthesis on a chemical synthesizer (1), the chemical synthesizer comprising an automated chemical reactor (12), a communication module (13), and a control module (11), the method comprising receiving, in the control module (11) using the communication module (13), a recipe message from a blockchain platform, the recipe message comprising an encrypted chemical synthesis recipe; decrypting, in the control module (11), the encrypted chemical synthesis recipe using a cryptographic key securely stored in the control module (11); and performing, in the automated chemical reactor (12), the chemical synthesis according to the chemical synthesis recipe.

### FIELD OF THE DISCLOSURE

The present disclosure relates to methods and devices for performing a chemical synthesis on a chemical synthesizer.

### BACKGROUND OF THE DISCLOSURE

A chemical reaction involves supplying one or more chemical reagents the necessary activation energy for the reagents to break and make chemical bonds to form new chemical entities, which are termed as product(s) and/or by-product(s) of the reaction. Chemical reactions are often carried out in a reactor of a chemical synthesizer.

Various factors, such as reagent flowrate, residence time, reagent concentration/equivalent, reaction temperature, pressure, pH, design of the reactor (to name a few), influence the reaction mechanism and kinetics of chemical reaction, which influences the outcome of the reaction, either measured by the yield and stereo-/regio-selectivity of the desired product or by shape, size, morphology and properties of particles, in case of synthesis of micro- and nanoparticles in the synthesizer.

Synthesizing products and by-products from the chemical reaction involves various steps, such as mixing the reagents, heating the reactor to the desired reactor temperature, introducing the reagents at a certain flow rate into the reactor, keeping the reagent continuously flowing through the reactor until certain time, called ‘residence time’ or ‘reaction time’, and collecting the reaction fluid from the reactor. These sequences of steps are specific to a particular reaction and may be adjusted or tweaked such that the desired outcome of the reaction is achieved.

These steps can be programmed into an automated chemical synthesizer which can carry out these steps automatically, i.e. with little or no human intervention. The automated chemical synthesizer can contain various modules such as a reagent delivery module and a reactor modules. Additional monitoring and analytical modules may also be provided to monitor the chemical synthesizer, monitor the reaction itself, or analyse the outcome of the reaction.

The data provided by the modules, such as the flowrate of the reagents, concentrations of reagents, reaction temperature, residence time, and output from the analytical instruments to calculate yield and selectivity towards the desired product, respectively the entirety of the input, output and process data are specific to the reaction. Using the aforementioned data, chemical synthesis recipes resp. unique reaction fingerprints (URFs) can be computed which provide a complete log of information regarding the chemical synthesis. The URF represents an image of the chemical synthesis.

Since the release of Bitcoin, further blockchain platforms have proliferated which have enabled not only the establishment of further cryptocurrencies but have provided decentralized platforms for providing more features and functions. Blockchain platforms may be generally described as decentralized databases with a consensus mechanism used to commit changes to the database. Depending on the particular blockchain platform, various computational capabilities can also be provided allowing for the decentralized execution of software.

### SUMMARY OF THE DISCLOSURE

It is an object of the present disclosure to provide methods and computer program products for performing a chemical synthesis on a chemical synthesizer, which overcomes one or more disadvantages of the prior art.

According to the present disclosure, the above-mentioned objects are achieved by a method for performing a chemical synthesis on a chemical synthesizer. The chemical synthesizer comprises an automated chemical reactor, a communication module, and a control module. The control module is connected to the automated chemical reactor and the communication module. The method comprises receiving, in the control module using the communication module, a recipe message. The recipe message is received from a blockchain platform. The recipe message comprises an encrypted chemical synthesis recipe. The method comprises decrypting, in the control module, the encrypted chemical synthesis recipe using a cryptographic key. The method comprises performing, in the automated chemical reactor, the chemical synthesis according to the chemical synthesis recipe.

Thereby, the chemical synthesis is performed fully automatically by the chemical synthesizer according to the received recipe message. The control module obtains the recipe message from the blockchain platform and performs, using the chemical reactor, the chemical synthesis. The chemical synthesis recipe is not necessarily stored on the chemical synthesizer prior to the recipe message being received, and may be only temporarily stored in the chemical synthesizer during chemical synthesis (e.g., in volatile memory), and then afterwards discarded, deleted, rendered unreadable or otherwise rendered unusable. Thereby, confidentiality of the details of the chemical synthesis recipe can be maintained. An originator or creator of the chemical synthesis recipe can provide the chemical synthesis recipe, using the blockchain platform, to the chemical synthesizer, while maintaining confidentiality of the details of the chemical synthesis recipe.

In an embodiment, the cryptographic key to decrypt the encrypted chemical synthesis recipe is an asymmetric key. An asymmetric key is part of a private and public key pair, wherein encryption is performed using one of the keys and decryption is performed using the other key. For example, the private key is used to decrypt the encrypted chemical synthesis recipe, the chemical synthesis recipe having been encrypted using the public key.

In an embodiment, the cryptographic key to decrypt the encrypted chemical synthesis recipe is a symmetric key. A symmetric key is a key that can be used for encrypting and decrypting in a same instance. In other words, the encryption key and the decryption key are the same. The symmetric key may be received by the chemical synthesizer, from a key server, in an encrypted fashion, in particular encrypted, by the key server, using a public key of the chemical synthesizer previously transmitted to the key server.

The cryptographic key is preferably stored securely in the chemical synthesizer.

In an embodiment, the chemical synthesizer further comprises an input module. The method further comprises receiving, in the control module via the input module, an input indicative of a reaction identifier. The method comprises transmitting, by the control module using the communication module, to the blockchain platform, a selection message indicative of the reaction identifier. The method comprises receiving, by the control module using the communication message, the recipe message associated with the reaction identifier.

In an embodiment, the reaction identifier is created according to a blockchain algorithm or a random number generator.

In an embodiment, the method further comprises generating, in the control module, a confirmation message. The confirmation message comprises a reaction identifier of the chemical synthesis recipe and a chemical synthesizer identifier. The confirmation message is indicative of an initiated chemical synthesis. The method comprises transmitting, by the control module using the communication module, the confirmation message to the blockchain platform.

In an embodiment, the method further comprises generating a timestamp and the confirmation message comprises the timestamp.

In an embodiment, the method comprises generating and transmitting a completion confirmation message indicative of a completed chemical synthesis.

In an embodiment, the chemical synthesis recipe further comprises indicators of control parameters of a chemical synthesis in a chemical synthesizer. The indicators of control parameters may comprise indicators of reagents, process factors, and/or process steps. The indicators of process factors may comprise a reagent concentration, a reagent pH, pKa, and/or design of the automated chemical reactor. The indicators of process steps may comprise residence time, reaction temperature profile, reagent flowrate profile, reaction pressure profile, and/or collection time.

In an embodiment, the method further comprises logging, in the control module, control parameters of the chemical synthesizer during chemical synthesis. The control module preferably logs the control parameters in real-time. The method comprises recording, by the control module, control parameter data generated from the logged control parameters.

In an embodiment, the method further comprises adjusting the chemical synthesis recipe according to user input received in the chemical synthesizer. Thereby, the control parameters logged by the chemical synthesizer may deliberately deviate from the indicators of control parameters as defined by the chemical synthesis recipe. Additionally, or alternatively, the method may comprise tuning the chemical synthesis recipe using an optimizing function. The tuning function may be configured to deterministically and/or stochastically generate one or more tuned control parameters.

In an embodiment, the chemical synthesizer further comprises an analytics module.

The method further comprises analyzing, using the analytics module, reaction products of the chemical synthesis. The method comprises generating, in the analytics module, reaction product data based on the analyzed reaction products. The method comprises recording, by the control module, reaction product data from the analytics module.

In an embodiment, the method further comprises generating, by the control module, a reaction report message including the control parameter data and/or the reaction product data. The method comprises encrypting, by the control module, the reaction report message. The method comprises transmitting, by the control module using the communication module, the report message to the blockchain platform.

In an embodiment, the method further comprises generating, in the control module, a new chemical synthesis recipe using the received chemical synthesis recipe and the recorded control parameter data. The method comprises encrypting, in the control module, the new chemical synthesis recipe. The method may comprise generating the confirmation message to further include the encrypted new chemical synthesis recipe.

The new chemical synthesis recipe may be encrypted using the cryptographic key used to decrypt the encrypted chemical synthesis recipe. Alternatively, the new chemical synthesis recipe may be encrypted using a further cryptographic key different to the cryptographic key used to decrypt the encrypted chemical synthesis recipe.

In an embodiment, the method further comprises encrypting, in the control module, the reaction product data.

In an embodiment, the analysis module of the chemical synthesizer includes a High Performance Liquid Chromatography (HPLC) analysis unit, a Gas Chromatography (GC) analysis unit, a Gas Chromatography and Mass Spectroscopy (GC-MS) analysis unit, a Liquid Chromatography and Mass Spectroscopy (LC-MS) analysis unit, a Mass Spectroscopy (MS) analysis unit, a Nuclear Magnetic Resonance (NMR) analysis unit, a Ultraviolet-Visible (UV-Vis) analysis unit, and/or an Infrared (IR) analysis unit or other process analytical technologies (PATs).

In an embodiment, the chemical synthesizer further comprises a display module and the received recipe message further comprises reaction product data. The method further comprises displaying, with the display module, the reaction product data to a user.

In addition to the method for performing a chemical synthesis on a chemical synthesizer, the present disclosure further relates to a method for providing, by a source chemical synthesizer, a chemical synthesis recipe for use by a peer chemical synthesizer. According to the method, the source chemical synthesizer comprises at least one automated chemical reactor, at least one analytics module, a communication module, and a control module. The method comprises performing, in the source chemical synthesizer, a chemical synthesis in the automated chemical reactor according to a pre-defined chemical synthesis recipe. The method comprises analyzing, in the source chemical synthesizer, reaction products of the chemical synthesis using the analytics module. The method comprises generating, in the analytics module, reaction product data based on the analyzed reaction products. The method comprises receiving and recording, in the source chemical synthesizer, reaction product data from the analytics module. The method comprises encrypting, in the control module, the chemical synthesis recipe using a cryptographic key. The method comprises generating, in the control module, a reaction report message comprising the reaction product data. The method comprises transmitting, using the communication module, the reaction report message and the encrypted chemical synthesis recipe to a blockchain platform for use by the peer chemical synthesizer.

In an embodiment, the analysis module of the source chemical synthesizer includes one or more of: a HPLC analysis unit, GC analysis unit, GC-MS analysis unit, LC-MS analysis unit, MS analysis unit, NMR analysis unit, UV-VIS analysis unit, or IR analysis unit.

In addition to the methods disclosed herein, the present disclosure also relates to a chemical synthesizer. The chemical synthesizer comprises at least one automated chemical reactor, a communication module and a control module. The control module is connected to the automated chemical reactor and the communication module. The control module is configured to receive, using the communication module, a recipe message from a blockchain platform. The recipe message comprises an encrypted chemical synthesis recipe. The control module is configured to decrypt the encrypted chemical synthesis recipe using a cryptographic key. The control module is configured to perform, using the automated chemical reactor, the chemical synthesis according to the chemical synthesis recipe.

In addition to the methods and the chemical synthesizer disclosed herein, the present disclosure also relates to a source chemical synthesizer configured to provide an encrypted chemical synthesis recipe for use by a peer chemical synthesizer. The source chemical synthesizer comprises an automated chemical reactor, an analytics module, a communication module, and a control module. The control module is configured to perform a chemical synthesis in the automated chemical reactor according to a predefined chemical synthesis recipe. The control module is configured to analyze reaction products of the chemical synthesis using the analytics module. The control module is configured to generate, in the analytics module, reaction product data based on the analyzed reaction products. The control module is configured to receive and record the reaction product data from the analytics module. The control module is configured to encrypt, in the control module, the chemical synthesis recipe using a cryptographic key.

The control module is configured to generate a reaction report comprising the reaction product data. The control module is configured to transmit, using the communication module, the reaction report and the encrypted chemical synthesis recipe to a blockchain platform, in particular for use by a peer chemical synthesizer, in particular for use in performing the chemical synthesis recipe.

The present disclosure also relates to a computer program product. The computer program product comprises computer program code configured to direct a control module of a chemical synthesizer to perform a method as disclosed herein, in particular for performing a chemical synthesis on a chemical synthesizer.

The present disclosure also relates to a further computer program product. The further computer program product comprises computer program code configured to direct a control module of a source chemical synthesizer to perform a method as disclosed herein, in particular for providing, by the source chemical synthesizer, a chemical synthesis recipe for use by a peer chemical synthesizer.

The present disclosure also relates to a non-transitory computer readable medium having stored thereon computer program code configured to direct a control module of a chemical synthesizer to perform a method as disclosed herein, in particular for performing a chemical synthesis on a chemical synthesizer.

The present disclosure also relates to a further non-transitory computer readable medium having stored thereon computer program code configured to direct a control module of a source chemical synthesizer to perform a method as disclosed herein, in particular for providing, by the source chemical synthesizer, a chemical synthesis recipe for use by a peer chemical synthesizer.

### BRIEF DESCRIPTION OF THE DRAWINGS

The herein described disclosure will be more fully understood from the detailed description given herein below and the accompanying drawings, which should not be considered limiting to the invention described in the appended claims. The drawings in which:

FIG. 1 shows a block diagram schematically illustrating a peer chemical synthesizer for performing a chemical synthesis;

FIG. 2 shows a block diagram schematically illustrating a source chemical synthesizer for providing a chemical synthesis recipe;

FIG. 3 shows a block diagram schematically illustrating a recipe message comprising a chemical synthesis recipe;

FIG. 4 shows a block diagram schematically illustrating a confirmation message;

FIG. 5 shows a block diagram schematically illustrating a blockchain platform for storing chemical synthesis recipes and exchanging these recipes between source chemical synthesizers and peer chemical synthesizers;

FIG. 6 shows a flow diagram illustrating a number of exemplary steps for performing a chemical synthesis on a chemical synthesizer;

FIG. 7 shows a flow diagram illustrating a number of exemplary steps for performing a chemical synthesis on a chemical synthesizer;

FIG. 8 shows a flow diagram illustrating a number of exemplary steps for performing a chemical synthesis on a chemical synthesizer; and

FIG. 9 shows a flow diagram illustrating a number of exemplary steps for providing, by a source chemical synthesizer, a chemical synthesis recipe to blockchain platform for user by a peer chemical synthesizer.

### DESCRIPTION OF THE EMBODIMENTS

Reference will now be made in detail to certain embodiments, examples of which are illustrated in the accompanying drawings, in which some, but not all features are shown.

Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

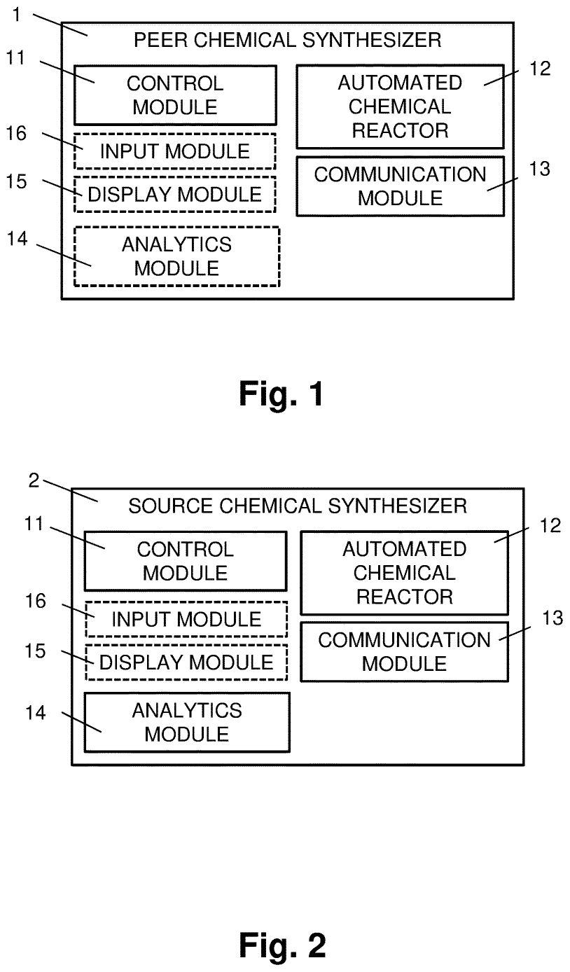

FIG. 1 shows a block diagram schematically illustrating a chemical synthesizer 1 for performing a chemical synthesis. The chemical synthesizer may be referred to as a peer chemical synthesizer 1 herein. The peer chemical synthesizer 1 comprises a control module 11, an automated chemical reactor 12, a communication module 13, and optional further modules as described herein. The control module 11 is connected to the automated chemical reactor 12 and the communication module 13.

The term chemical synthesis as used herein refers to all types of chemical synthesis, including but not limited to synthesis reactions, decomposition reactions, single and/or double displacement reactions, forward and backward reactions, combustion, redox, complexation reactions, acid-base reactions, precipitation reactions, solid-state reactions, and photochemical reactions. Organic chemistry reactions and biochemical reactions are also included, along with reactions including catalysts.

The automated chemical reactor 12 is a standard automated chemical synthesis reactor as known in the chemical and pharmaceutical industries and may be used, for example, for production, as well as for research and development. The automated chemical reactor 12 may be designed as a batch reactor, a continuous stirred-tank reactor, and/or a plug flow reactor. As such the automated chemical reactor 12 includes one or more volumes in which chemical reactions take place, along with actuators for controlling the automated chemical reactor 12 (e.g. valves, agitators, pumps, thermal actuators (including heating units and cooling units)). The automated chemical reactor 12 may further include sensors for monitoring the automated chemical reactor 12 itself and/or monitoring the reaction taking place inside. The sensors may include one or more temperature sensors, one or more flow sensors, one or more pressure sensors, etc.

The automated chemical reactor 12, in particular the actuators and optionally the sensors, are controlled by the control module 11. The control module 11 is not necessarily entirely separated from the automated chemical reactor 12. In particular, one or more methods, steps, functions or parts thereof which are described herein as being performed by the control module 11 may be performed by the automated chemical reactor 12. The control module 11 controls the automated chemical reactor 12 such that the automated chemical reactor 12 performs a chemical synthesis according to chemical synthesis recipe. The control module 11 is provided with the chemical synthesis recipe as described herein. The chemical synthesizer 1 is therefore configured to perform automatic chemical synthesis, in particular without substantial human control or intervention during the synthesis. Therefore, chemical synthesis can be performed largely unattended. Additionally, data collected by the sensors may be automatically stored.

The automated chemical reactor 12 may include one or more inputs, including inlets for fluid (i.e., liquid or gas) or particulate input, reagent storage vessels, etc. These inputs are configured to store and/or provide reagents, precursors, and/or catalysts to the automated chemical reactor 12 for the chemical synthesis. The automated chemical reactor 12 may further include one or more outputs, including outlets for fluid or particulate output, output storage vessels etc. These outputs are configured to receive the products and/or byproducts of the chemical synthesis.

In an embodiment, the chemical synthesizer 1 comprises multiple automated chemical reactors 12. These multiple automated chemical reactors 12 may be configured and/or used for different synthesis steps or for the same synthesis step in parallel. The provision of reagents, potential solvents and/or products from a given automated chemical reactor 12 to another automated chemical reactor 12 may be performed by a user, for instance in that the control module 11 of the chemical synthesizer 1 notifies the user with a display module 15 that this provision needs to be performed and the user confirms to the chemical synthesizer 1, 2 resp. the control module 11 via an optional input module that the provision of reagents and/or products has been performed. Additionally and alternatively, the chemical synthesizer 1 further comprises transportation means, which may include one or more actuators, that are configured to perform the step of provisioning reagents, potential solvents and/or products from an archive of available reagents and potential solvents to an automated chemical reactor 12 or from the given automated chemical reactor 12 to another automated chemical reactor 12. The transportation means may include passive means for connecting the given automated chemical reactor 12 to one or more further automated chemical reactors 12, the passive means comprising, for example, hoses, tubes, and/or other fluid transportation means. The transportation means may include actuators, for example in the form of robotic appliances including motors and/or robotic arms capable of moving laboratory glassware as flasks, cylinders, beakers and/or pipettes.

In an embodiment, the chemical synthesizer 1 may comprise multiple analysis modules 14. Either the user and/or transportation means can perform the step of provisioning products from a given automated chemical reactor 12 to an analysis module 14 or different portions of the product may be provisioned from a given automated chemical reactor 12 to different analysis modules 14 and analyzed in parallel. Alternatively or additionally, products may also be provisioned from a given analysis module 14 to another analysis module 14.

In an embodiment, the automated chemical reactor 12 further comprises equipment to cool or heat parts of the reactants and/or stir the reactants, for instance hot plates, jacketed vessels, magnetic stirrers and/or overhead stirring.

The control module 11 comprises a processor. The control module 11 further comprises a memory. For sake of simplicity, the control module 11 is described as separate from the other components of the chemical synthesizer 11, such as the automated chemical reactor 12 and the communication module 13. However, the skilled person understands that the control module 11 and the other components may have at least some degree of integration on the hardware and/or software level. Further, at least some of the methods, steps or functionality described herein as being performed by the control module 11 may be performed by other components of the chemical synthesizer 11.

The processor of the control module 11 is configured to perform the methods, steps and/or functions as described herein, in part or in whole, by execution of computer program code stored in the memory. The processor may comprise a system on a chip (SoC), a central processing unit (CPU), and/or other more specific processing units such as a graphical processing unit (GPU), application specific integrated circuits (ASICs), or reprogrammable processing units such as field programmable gate arrays (FPGAs). Additionally, the processor may comprise additional processing units specifically configured to accelerate certain applications, such as cryptographic accelerators for accelerating cryptographic functions.

The memory of the control module 11 comprises one or more volatile (transitory) and/or non-volatile (non-transitory) storage components. Examples of storage components include RAM (Random Access Memory), flash memory, data memory, and/or other data stores. The memory has stored therein program code configured to control the processor of the control module 11, such that the control module 11 performs one or more steps and/or functions as described herein. Depending on the embodiment, the program code is compiled or non-compiled program logic and/or machine code. The memory stores, in general, both computer program code and data. The computer program code is configured to control the processor of the control module 11 to execute a series of steps and/or functions.

The computer program code defines and/or is part of a discrete software application. One skilled in the art will understand that the computer program code can also be distributed across a plurality of software applications (Apps). In an embodiment, the computer program code further provides interfaces, such as APIs, such that functionality and/or data of the chemical synthesizer 1 can be accessed remotely, such as via an external controlling device, such as a connected client computer, via client application, or via a web browser.

Depending on the embodiment, the chemical synthesizer 1, in particular the control module 11, comprises additional submodules, for example a power module. The power module comprises a battery and/or power receiving means, such as a power socket for receiving electrical power via a wired connection and/or a loop antenna for receiving electrical power via magnetic induction.

In an embodiment, the control module 11, or parts of the control module 11 are secured from unauthorized access, for example by the use of a tamper resistance hardware security module such as a secure cryptoprocessor. The secure cryptoprocessor may be implemented as a physical or virtual smartcard and/or a trusted platform module. The hardware security module may be implemented in separate hardware from the processor. The hardware security module may, alternatively, be integrated into the processor.

The hardware security module includes processing circuitry for securely executing specific computer program code and includes secure memory for securely storing the specific computer program code, as well as sensitive data. The sensitive data includes, for example, a cryptographic key. The cryptographic key used for securing data and communication may be initially installed and stored in the secured memory during manufac-ture or commissioning of the control module 11. The processor of the control module 11 is configured to control the hardware security module, for example by calling one or more specific functions, such that the hardware security module performs one or more steps or functions as described herein. For example, the specific computer program code further includes one or more cryptographic functions, the one or more cryptographic functions comprising an encryption function, a decryption function, a hash function, a cryptographic key generating function, a cryptographic key deletion function, a cryptographic signing function, and/or a cryptographic signature verification function. In an embodiment, the functions performed by the hardware security module further comprise a random number generator function. The random number generator function is configured to control the processor to generate random or pseudo-random numbers. In an embodiment, the control module 11 further includes a hardware-based random generator which generates true random numbers.

The communication module 13 is a hardware module configured for wired or wireless communication with a blockchain platform 6. In particular, the communication module 13 is configured to communicate with one or more nodes of the blockchain platform, either directly or indirectly. The communication may take place over one or more intermediary communication networks, for example including the Internet. The communication network may also include a local area network (LAN), a wireless local area network (WLAN), and/or a mobile radio network, such as a GSM-network (Global System for Mobile communication), a UMTS-network (Universal Mobile Telephone System) or another mobile radio telephone system.

The communication module 13 may communicate with the blockchain platform 6 using an intermediary service, such as a remote procedure call (RPC) node, which is a server computer that enables the control module 11, using the communication module 13, to read data on the blockchain platform 6 and send messages (e.g., in the form of transactions) to the blockchain platform 6.

Depending on the embodiment, the control module 11 is configured to implement, or act as, a node on the blockchain platform 6. The control module 11 may be configured to implement a full node, or a light node. The full node replicates the entire blockchain of the blockchain platform 6, in particular a main chain of the blockchain platform 6, while the light node replicates only a part of the entire blockchain of the blockchain platform 6. The control module 11 may additionally or alternatively interact with the blockchain platform 6 via one or more intermediary devices, which intermediary devices may implement a node (e.g., a full node or a light node) of the blockchain platform 6.

The control module 11 may have an assigned address on the blockchain platform 6. The address may be derived from, or using, a public key. The public key is associated with the particular chemical synthesizer 1 and communicated, via a communication network, to other nodes on the blockchain platform 6. These other nodes may correspond to other chemical synthesizers 1 on the blockchain platform 6. Other chemical synthesizers 1, however, may also interact with the blockchain platform 6 via intermediary devices and/or services. The control module 11 may additionally have a private key, the private key being associated with its public key according to public key cryptography. The private key is preferably securely stored in the control module, for example in the secure cryptoprocessor.

The private key may be used to digitally sign transactions sent by, or on behalf of, the particular chemical synthesizer 1. Receiving devices may verify the validity of the transaction using the public key.

The control module 11 may also store public keys of one or more other chemical synthesizers 1 and use these public keys, for example, to encrypt transactions directed to the one or more other chemical synthesizers 1, respectively.

The control module 11 may additionally generate and/or establish a symmetric (cryptographic) key with another chemical synthesizer using public key cryptography. The symmetric key may be used for subsequent communication, in particular for encrypting and decrypting particular transactions or parts thereof.

The control module 11 may additionally be configured to authenticate the other components of the chemical synthesizer 1, in particular the automated chemical reactor 12. For example, the control module 11 may have stored (e.g., stored in the secure cryptoprocessor), an allow-list indicative of one or more allowed components. The allow list may include, for example a list of allowed identifiers, such as defined using a hardware or physical address, and/or as defined using one or more cryptographic keys. During start-up of the chemical synthesizer 1, for example after powering the chemical synthesizer 1 on, the chemical synthesizer 1 may authenticate connected components by requesting and/or receiving, from the components, an identifier and/or a digitally signed message.

The chemical synthesizer 1 is configured to proceed only upon affirmative authentication of the connected components. Thereby, the chemical synthesizer 1 may improve the security and/or reliability of its operation by ensuring only legitimate components are currently installed.

In an embodiment, the chemical synthesizer 1 comprises an input module 16 and/or a display module 15, which are connected to the control module 11. The input module may be any hardware module that allows a user to input data into the chemical synthesizer 1 and may include a keyboard, a mouse, a touch-screen, etc. The display module 15 may be any hardware module that allows a user to receive information from the chemical synthesizer, such as a screen.

In an embodiment, the peer chemical synthesizer 1 comprises an analytics module 14.

The analytics module 14 is configured to analyze reaction produces produced by the automated chemical reactor 12. The analytics module 14 includes, for example, a high performance liquid chromatography (HPLC) analysis unit, a gas chromatography (GC) analysis unit, a gas chromatography and mass spectrometer (GC-MS) analysis unit, a liquid chromatography and mass spectrometer (LC-MS) analysis unit, a mass spectrometer (MS) analysis unit, a nuclear magnetic resonance (NMR) analysis unit, a ultraviolet and visible (UV-VIS) and/or an Infrared (IR) analysis unit. In particular, the analytics module 14 is configured to analyze the reaction products output by the automated chemical reactor 12. The analytics module 14 is configured to generate reaction product data using based on the analyzed reaction products and provide the reaction product data to the control module 11.

FIG. 2 shows a block diagram schematically illustrating a chemical synthesizer 2 configured to provide a chemical synthesis recipe. The chemical synthesizer 2 may be designated as a source chemical synthesizer 2, in that it defines a source of information, in particular a source of a chemical synthesis recipe. The chemical synthesizer 2 provides the chemical synthesis recipe to one or more further chemical synthesizers, which may be designated as peer chemical synthesizers 1. A given chemical synthesizer 1, 2 may be, or act as, a peer chemical synthesizer 1 at a given time-point, and be, or act as, a source chemical synthesizer 2 at a different time-point. The given chemical synthesizer 1, 2, may act both as a source and peer simultaneously. The source chemical synthesizer 2 includes the same components as the peer chemical synthesizer 1 described above with reference to FIG. 1, however the analytics module 14 is mandatory.

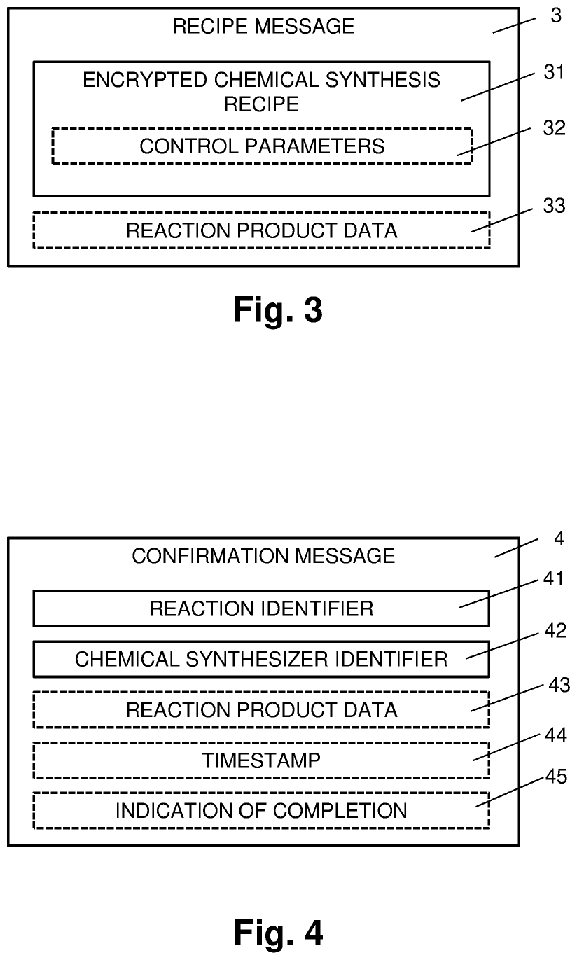

FIG. 3 shows a block diagram schematically illustrating a recipe message 3. The recipe message 3 is transmitted or otherwise provided to the blockchain platform and then stored on the blockchain platform. The recipe message 3 may be transmitted to the blockchain platform by a source chemical synthesizer 2 as described herein.

The recipe message 3 may alternatively be transmitted to the blockchain platform by another source device. The recipe message 3 may be stored on the blockchain platform as part of a transaction. For example, the recipe message 3 may be stored on a public blockchain such as Ethereum by including, in a transaction, the recipe message 3 in a data field. The transaction may be directed to a particular cryptographic address on the blockchain or to a smart contract, for example.

The recipe message 3 is retrieved from the blockchain platform by the (peer) chemical synthesizer 1 in order to perform the chemical synthesis.

The recipe message 3 comprises an encrypted chemical synthesis recipe 31. The chemical synthesis recipe 31 is encrypted such that it cannot be read or interpreted without decryption using an appropriate cryptographic key. The chemical synthesis recipe 31 may be formatted as a binary file.

The chemical synthesis recipe 31 provides information necessary for the chemical synthesizer 1 to perform the chemical synthesis.

The recipe message 3 and/or the chemical synthesis recipe 31 may include information which defines the chemical reaction(s) performed during the chemical synthesis, the chemical reactant(s) and/or agent(s) (e.g. a chemical fingerprint of the chemical reactant(s) and/or agent(s)), a chemical fingerprint of the product(s), the chemical fingerprint of the reactant side of the reaction center (including, optionally, its 1 bond or 2 bond neighbor), the chemical fingerprint of the product side of the reaction center (including, optionally, its 1 bond or 2 bond neighbor). The information which defines the chemical reaction(s) may be include a reaction fingerprint, such as an Extended Connectivity Fingerprint (ECFP), a chemical hashed fingerprint, a pharmacophore fingerprint (PF), a reaction fingerprint (RF), and/or a ChemAxon MACCS-166 fingerprint.

The chemical synthesis recipe may include, for example, control parameters 32 of the chemical synthesis. The control parameters 32 may include settings for one or more actuators of the chemical synthesizer 1. The settings may be time-varying. For example, the control parameters 32 may indicate a heating signal for controlling a reactor heater.

The control parameters 32 may further include one or more set-point values as defined by particular sensor values, such as a time-varying temperature set-point value which defines a reactor temperature.

The recipe message 3 may further include indicators of required reagents, catalysts, and/or precursors. The recipe message 3 may further include indicators of one or more additional hardware modules of the chemical synthesizer 1, or a particular configuration of the chemical synthesizer 1, required for the particular chemical synthesis.

The recipe message 3 may further include, or have associated therewith, a description of the chemical synthesis, for example in the form of a reaction report message which may include reaction product data 33. The description may include one or more products produced by the chemical synthesis, including details related to the yield, purity, byproducts, etc.

The recipe message 3 is described herein as being stored on the blockchain platform. This may include but is not limited to the main-chain. In particular, the blockchain platform may include one or more sidechains. The blockchain platform may further include a data store, for example a decentralized data store, configured for file storage.

FIG. 4 shows a block diagram schematically illustrating a confirmation message 4.

The confirmation message 4 is generated by the chemical synthesizer 1 upon initiation of the chemical synthesis. The confirmation message 4 thereby indicates that the chemical synthesizer has initiated chemical synthesis. Depending on the embodiment, the confirmation message 4 may be generated immediately prior to chemical synthesis, during chemical synthesis, and/or upon completion of the chemical synthesis.

The confirmation message 4 comprises a reaction identifier 41. The reaction identifier 41 identifies the chemical reaction recipe. The reaction identifier may be generated by the chemical synthesizer 1, or included in the recipe message 3.

The confirmation message 4 further comprises a chemical synthesizer identifier 42 which uniquely identifies the chemical synthesizer 42.

In an embodiment, the confirmation message 4 further comprises reaction product data 43 related to the reaction products. The confirmation message may comprise a timestamp 44 and/or an indication of completion 45 of the chemical synthesis.

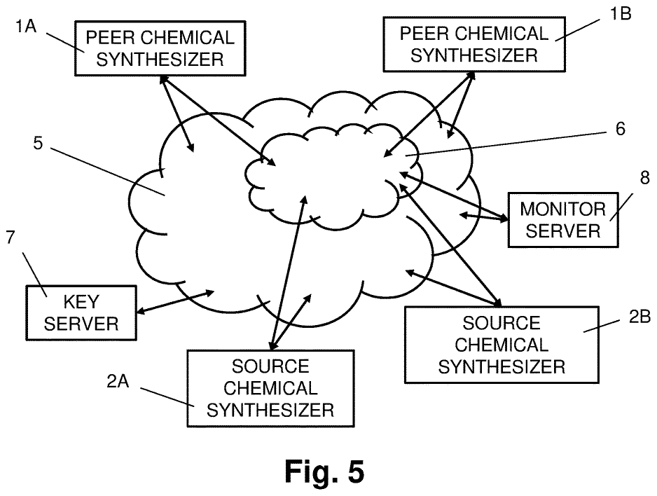

FIG. 5 shows a block diagram schematically illustrating a communication network 5 (which may include the Internet) and a blockchain platform 6, along with peer chemical synthesizers 1A, 1B and source chemical synthesizers 2A, 2B communicatively connected via the blockchain platform 6. The blockchain platform 6 includes a plurality of nodes, which are computers which execute appropriate software to implement the particular blockchain, which computers are communicatively connected to each other and exchange transactions, such that a decentralized database (also referred to as a ledger) is established and maintained. The blockchain platform 6 may be a public blockchain platform 6, such as Bitcoin or Ethereum. The blockchain platform 6 may alternatively or additionally include a private blockchain platform 6, for example Hyperledger fabric, Ripple, etc. Other examples of blockchain platforms include IBM blockchain, Hyperledger, Sawtooth, and R3 Corda. The blockchain platform 6 may be permissionless, meaning that any node with suitable software may join the blockchain platform, or permissioned, in which nodes require authentication.

The blockchain platform 6 includes a main chain. The blockchain platform 6 may additionally include one or more side chains. The blockchain platform 6 may additionally include one or more data stores, for example decentralized data stores.

The blockchain platform 6 stores recipe related data, for example one or more encrypted chemical synthesis recipes 31 included in one or more recipe messages 3. In particular, one or more nodes of the blockchain platform 6 stores the one or more encrypted chemical synthesis recipes 31. The blockchain platform 6 may also store other recipe related data such as a decryption key. The blockchain platform 6, in particular a source chemical synthesizers 2A, 2B, transmits a confirmation message 4 to the blockchain platform 6 and the peer chemical synthesizers 1A, 1B receive a recipe message 3 from the blockchain platform 6. Thereby, the blockchain platform 6 facilitates an exchange of encrypted chemical synthesis recipes 31 between source chemical synthesizers 2 and peer chemical synthesizers 1.

In an embodiment, a blockchain platform specific consensus mechanism, one or more smart contracts and/or some form of private communication channel are used before a peer chemical synthesizers 1 receives recipe related data. The recipe related data is for example a recipe message 3 comprising an encrypted chemical synthesis recipe 31 or a decryption key. The specific type and/or configuration of the smart contract(s) and/or private communication channel may depend on the type of blockchain platform 6 used.

Thereby, a securely stored encrypted chemical synthesis recipe 31 may be transmitted, as part of a recipe message 3 comprising the encrypted chemical synthesis recipe 31, to a peer chemical synthesizer 1A, 1B via the blockchain platform 6.

In an embodiment, the peer chemical synthesizer 1A, 1B, for example upon receiving user input from a user of a peer chemical synthesizer 1A, 1B, may request to receive a chemical synthesis recipe in order to perform it. The consensus mechanism among the nodes of the blockchain platform 6 implemented on the blockchain platform 6 may approve and therefore allow the transmittal of the recipe related data according to the chemical synthesis recipe. The consensus mechanism used by the nodes of the blockchain platform 6 may include at least one of the following: proof of work (PoW), proof of stake (PoS), or proof of capacity (PoC). These mechanisms may be used for so-called permissionless blockchain platforms 6. In the case of permissioned blockchain platforms, the consensus mechanism may include a voting mechanism in which one or more nodes, in particular a rotating subset of nodes, or a particular proportion of nodes, such as a majority of nodes, approve a new block.

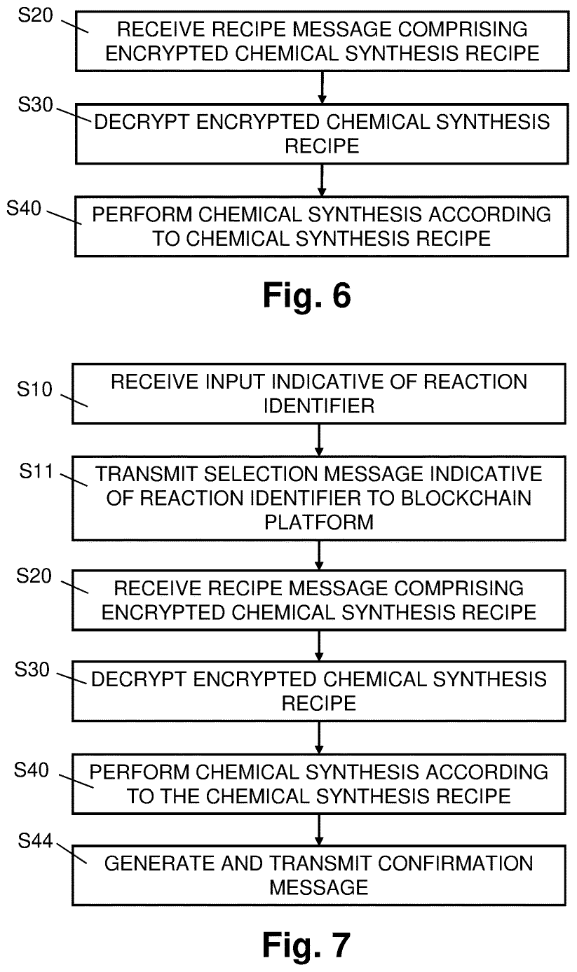

FIG. 6 shows a flow diagram illustrating a method including a number of steps for performing a chemical synthesis on a chemical synthesizer 1.

In a step S20 the peer chemical synthesizer 1 receives, in the control module 11, a recipe message 3 comprising an encrypted chemical synthesis recipe 31 from a blockchain platform 6.

Receiving the recipe message 3 may include the peer chemical synthesizer 1 accessing and/or reading a particular transaction included in the blockchain platform 6. In an embodiment, the peer chemical synthesizer 1, in particular the control module 11, implements a node of the blockchain platform 6. Thereby, the peer chemical synthesizer 1 has full access to all blocks of the main chain of the blockchain platform 6. In such a case, receiving the recipe message 3 may be understood as retrieving the recipe message 3 from the (local) node of the blockchain platform 6. Alternatively or additionally, the recipe message 3 may have been received previously, through the course of the (local) node of the blockchain platform 6 implemented on the peer chemical synthesizer 1 receiving, from other nodes of the blockchain platform 6, a particular transaction including the recipe message 3, and processing and storing the particular transactions in the memory.

The peer chemical synthesizer 1 may have an assigned address on the blockchain platform 6. The peer chemical synthesizer 1 may, depending on the implementation of the blockchain platform 6, have access only to transactions directed to the peer chemical synthesizer 1, in particular to the address of the peer chemical synthesizer 1.

In an embodiment, the peer chemical synthesizer 1, in particular the control module 11, receives the recipe message 3 from the blockchain platform 6 via an intermediary server (such as an RPC server).

In a step S30, the peer chemical synthesizer 1 decrypts, in the control module 11, the encrypted chemical synthesis recipe 31 included in the recipe message 3. The peer chemical synthesizer 1 may decrypt the encrypted chemical synthesis recipe 31 using a cryptographic key, for example using a cryptographic key securely stored in the control module 11.

The control module 11 may be configured to store the decrypted chemical synthesis recipe only in volatile, transient memory. Preferably, the control module 11 is configured such that the decrypted chemical synthesis recipe is protected, such that other applications or processes being performed by the control module 11 do not have access (in particular, cannot read) the chemical synthesis recipe.

The cryptographic key for decrypting the chemical synthesis recipe 31 may alternatively be received via a separate secure communication channel, for example from another chemical synthesizer 1 or a key server connected to the communication network. The cryptographic key may have been transmitted and/or received according to a smart contract on the blockchain platform 6.

The cryptographic key may be a symmetric key or an asymmetric key. Specifically, the cryptographic key used to decrypt the encrypted chemical synthesis recipe 31 may be identical (symmetric) to a cryptographic key used to encrypt the encrypted chemical synthesis recipe 31. The encrypted chemical synthesis recipe 31 may have been encrypted using asymmetric encryption (also known as public-key cryptography) and therefore be decrypted using a private key of the peer chemical synthesizer 1.

The cryptographic key may be securely stored in the secure cryptoprocessor. In an example, the control module 11 transmits the encrypted chemical synthesis recipe 31 to the secure cryptoprocessor for decryption, and receives the (unencrypted) chemical synthesis recipe 31 from the secure cryptoprocessor.

In a step S40, the chemical synthesis is performed in the automated chemical reactor 12 according to the chemical synthesis recipe. The control module 11 controls the automated chemical reactor 12 to perform the chemical synthesis as defined by the control parameters included in the chemical synthesis recipe. The control parameters include, for example, indicators of reagents, process factors, and/or process steps. The indicators of process factors may comprise a reagent concentration, a pH and/or a configuration of the automated chemical reactor 12. The indicators of process steps may comprise a residence time, a reaction temperature profile, a reagent flowrate profile, a reaction pressure profile, a concentration profile, a solvent profile, and/or a collection time.

For the performance of the chemical synthesis, some user intervention may be required, for example the provision of appropriate reagents, precursors and/or catalysts at the inputs of the automated chemical reactor 12 (depending on the precise reactor type and design). However, the user is not made aware of all details related to the process and therefore the chemical synthesis recipe remains confidential, at least in part.

In an embodiment, the control module 11 is configured for real-time control of the automated chemical reactor 12. In particular, the control module 11 is configured such that a control parameter and/or a set-point for a particular actuator and/or sensor of the automated chemical reactor 12, as defined by the chemical synthesis recipe for a particular time-point, is transmitted by the control module 11 at, or immediately prior to, that particular time-point. The automated chemical reactor 12 is preferably configured such that it does not, or cannot, log the control parameters and/or a set-points. Thereby, the automated chemical reactor 12 neither receives the entire chemical synthesis recipe at once, nor does it log or otherwise record the entire chemical synthesis recipe. Thereby, the security of the chemical synthesis recipe is improved and it cannot easily be obtained from the automated chemical reactor 12, for example by placing a device in between or by accessing a data store of the automated chemical reactor 12.

In the following passages, an example of how a particular chemical synthesis is performed will be described. The chemical synthesis is an exemplary reaction which produces rufinamide, an anticonvulsant drug. It is apparent that other chemical syntheses are conceivable to produce rufinamide.

For performing the synthesis of rufinamide, the control module 11 is configured for control, preferably real-time control, of the one or multiple automated chemical reactors 12 such that the chemical synthesizer 1 performs the synthesis shown in FIG. 10. The illustrated synthesis comprises three steps A, B and C, which produce rufinamide. The chemical synthesis producing rufinamide according to the three steps A, B and C as illustrated could be performed in one automated chemical reactor 12, but better results can be achieved with separate automated chemical reactors 12 for each synthesis step.

For example, the first automated chemical reactor 12, in which the first step A of the chemical synthesis takes place, may be required to be a tubular heated reactor, have a diameter of 1/16 inch, have a reactor volume of 10 ml and the heater have to be able to reach a tubular reactor temperature of 40° C. and keep the temperature steady for 5 minutes. The chemical synthesis recipe may include these requirements and an exemplary chemical synthesis recipe for step A including parameters as indicated is shown in Table 1 below.

TABLE 1

| Variable | Value | |
|---|---|---|
| Reactor type | tubular heated reactor | |
| Volume of reactor | 10 | ml |
| Reactor Temperature | 40° | C. |
| Reaction time | 5 | min |
| Reagent 1 | 2-(bromomethyl)-1,3-difluorobenzene | |
| Reagent 2 | Sodium azide | |
| Solvent | Acetonitrile and Water | |
| Flowrate of reagent pump 1 | 1 | ml/min |
| Flowrate of reagent pump 2 | 1 | ml/min |

The second and third steps B and C, respectively, of the chemical synthesis as shown in FIG. 10 may require second and third automated chemical reactors 12 with requirements different from those of the first automated chemical reactor 12. For instance, in the second step B of the synthesis, the reaction products of the first step A performed in the first automated chemical reactor are inserted, together with Copper(I)iodide and methyl propiolate, into the second automated chemical reactor 12 that may also have to be a tubular heated reactor, have a reactor diameter of 1/16 inch, have a reactor volume of 20 ml, and the heater have to be able to reach a tubular reactor temperature of 60° C. and keep the temperature steady for 20 minutes. Further, for instance, in the third step C of the synthesis, the reaction products of the second step B performed in the second automated chemical reactor are inserted, together with aqueous ammonia, into the second automated chemical reactor 12, that may also have to be a tubular heated reactor, have a reactor diameter of 1/16 inch, have a reactor volume of 20 ml, and the heater have to be able to reach a tubular reactor temperature of 60° C. and keep the temperature steady for 20 minutes, in order to produce rufinamide.

Analogous to the chemical synthesis recipe shown for step A above, these conditions may be encoded into a chemical synthesis recipe. The chemical synthesis recipe thereby may include conditions an automated chemical reactor 12 may have to fulfil, which reagents and solvents need to be inserted, and what flowrate is required for each of the reagents.

For simplicity's sake, the exemplary chemical synthesis recipe shown above does not comprise the steps of placing products of one step from an automated chemical reactor 12 to another automated chemical reactor 12 to perform a further step. However, such information may be included by the chemical synthesis recipe.

In an embodiment, the control module 11 outputs the required reactants and potential solvents to a display module 15, instructing a user to make them available. Alternatively and additionally, the control module 11 controls actuators to prepare the required reactants and potential solvents.

In an embodiment, in order to perform a chemical synthesis on a chemical synthesizer according to a chemical synthesis recipe, the control module 11 confirms the availability of the required reactants. The control module 11 may further confirm that the automated chemical reactor 12 fulfills the requirements indicated in the chemical synthesis recipe. If the required reactants and automated chemical reactors 12 are not available, the control module 11 may notify a user or a system via output means of the requirements that are not fulfilled.

In an embodiment, the control module 11 is further configured to initialize the parts of the chemical synthesizer according to the recipe.

The control module 11 may further notify a user and/or control robotic appliance(s) to place a product of a reaction from one automated chemical reactor 12 to another automated chemical reactor 12 and pause the synthesis until the user confirms having performed the requested step and/or the robotic appliance has performed the task.

FIG. 7 shows a flow diagram illustrating a number of exemplary steps for performing a chemical synthesis on a peer chemical synthesizer 1. In addition to the steps S20 to S40 described above with reference to FIG. 6, further steps may be performed. These steps may be performed if the recipe messages 3 are not currently available to the peer chemical synthesizer 1, for example if they are not yet available on the blockchain platform or if the chemical synthesizer 1 has not yet received the recipes messages 3 from the blockchain platform, however descriptions and/or other reaction identifiers of the chemical syntheses are available.

In a step S10, the control module 11 of the peer chemical synthesizer 1 receives, via the input module 16, an input indicative of a reaction identifier. For example, a user may select a particular reaction, the input of the user indicating one of a plurality of chemical reactions. In an optional preceding step, the peer chemical synthesizer 1 may retrieve, from the blockchain platform 6, a plurality of descriptions of chemical reactions, each description associated with a reaction identifier and/or a recipe message 3. The user may therefore select a chemical synthesis to be performed from among the plurality of chemical reactions.

In a step S11, the control module 11 transmits a selection message indicative of the reaction identifier to the blockchain platform 6 using the communication module 13. The selection message may be part of a transaction on the blockchain platform 6 from a sender address which corresponds to, or is assigned to, the peer chemical synthesizer 1. The transaction may be directed to a receiver address, which may be a smart contract.

The receiver address may further be the address on the blockchain platform 6 of a source chemical synthesizer 2.

Thereupon, the recipient of the selection message may transmit the recipe message 3, as part of a transaction, to the blockchain platform 6. For example, the transaction may be addressed to an address of the chemical synthesizer 1 on the blockchain platform.

Additionally or alternatively, the recipe message may be submitted via a separate secured communication channel to the chemical synthesizer 1.

The recipe message 3 received, for example from the blockchain platform 6 in step S20, comprises an encrypted chemical synthesis recipe 31, the encrypted chemical synthesis recipe 31 being associated with the reaction identifier. The association between the reaction identifier 41 and chemical synthesis recipe 31 may be realized in a number of ways, for example by a table which associates each chemical synthesis recipe 31 with a reaction identifier. Alternatively, the reaction identifier may be included as part of the chemical synthesis recipe 31. The reaction identifier generally uniquely identifies each chemical synthesis recipe.

In a step S44, a confirmation message 4 is generated in the control module 11. The confirmation message 4 may be generated immediately prior to performing the chemical synthesis, after initiation of the chemical synthesis (i.e., during performing of the chemical synthesis), and/or after completion of the chemical synthesis. The confirmation message 4 includes at least a reaction identifier 41 of the chemical synthesis and a chemical synthesis identifier 42. The confirmation message 4 may optionally include an indicator of a stage at which the chemical synthesis is (in terms of completion). For example, the confirmation message 4 may include an indicator of completion 45. The confirmation message 4 may further include a timestamp 44 generated by the control module 11, and/or a timestamp associated with the confirmation message 4 during a later step.

The confirmation message 4 is transmitted by the control module 11 to the blockchain platform 6. As mentioned above, a plurality of confirmation messages 4 may be transmitted by the peer chemical synthesizer 1 during chemical synthesis.

The blockchain platform 6 therefore receives, processes and/or stores the confirmation message 4, establishing a record that the particular peer chemical synthesizer 1 performed a chemical synthesis according to a particular chemical synthesis recipe. This record may be used for traceability or for maintenance purposes, for example for planning and scheduling maintenance for the peer chemical synthesizer 1 according to a number and/or a reaction type of chemical syntheses performed.

The blockchain platform 6 may further be monitored for confirmation messages 4 from a plurality of peer chemical synthesizers 1A, 1B. Thereby, a total number of chemical syntheses may be established, a total number of chemical syntheses of a particular reaction type may also be determined.

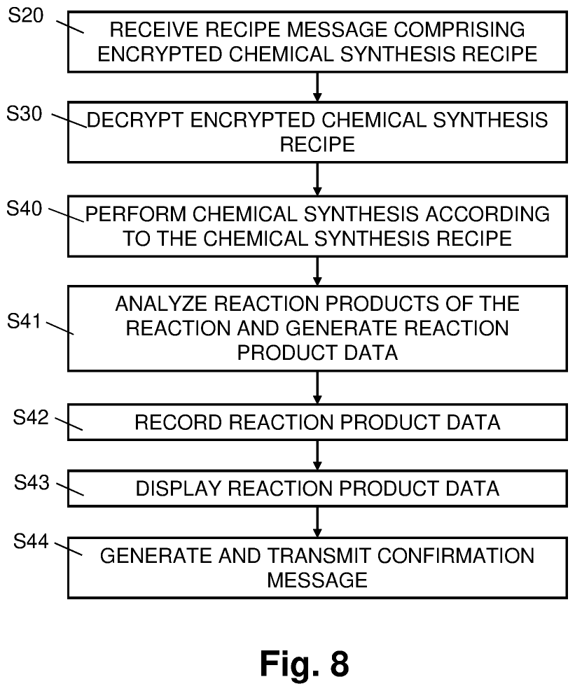

FIG. 8 shows a flow diagram illustrating a number of exemplary steps for performing a chemical synthesis on a peer chemical synthesizer 1. Steps S20 to S40 have been previously described with reference to FIG. 6. The peer chemical synthesizer 1, as described herein, may include an analysis module 14.

In a step S41, reaction products of the chemical synthesis are analyzed, using the analytics module 14. The reaction products may be analyzed during or subsequent to the chemical synthesis. The reaction products which are analyzed may include the main product, i.e. the desired result of the chemical synthesis, and may further include byproducts, i.e. secondary products which result incidentally from the main product.

The analytics module 14 is configured to generate reaction product data 43 based on the analyzed reaction products. This reaction product data 43 may include one or more types of chemicals produced during the chemical synthesis as well as parameters thereof or related thereto, including yield, purity, and/or concentration.

In a step S42, the reaction product data 45 is provided to the control module 11. For example, the reaction product data 45 is transmitted by the analytics module 14 and received by the control module 11, where it is recorded at least temporarily. The analysis and the recording of the reaction products may take place in real-time, simultaneous to performing the chemical synthesis in the automated chemical reactor 12 in step S40.

In an optional step S43, reaction product data is displayed on the display module 15.

In a step S44, the confirmation message 5 is generated in the control module 11 as described above with reference to FIG. 7. The confirmation message 5 is additionally generated to include the reaction product data 43. The confirmation message 5 thereby provides, to the blockchain platform 6, the reaction product data 43.

The blockchain platform 6 may be monitored to analyze the reaction product data 43 of one or more chemical syntheses. For example, the reaction product data 43 related to a particular peer chemical synthesizer 43 may be analyzed to determine whether the peer chemical synthesizer 43 is functioning according to specification, by comparing the reaction product data 43 of a particular chemical reaction with one or more defined tolerance thresholds. In another example the reaction product data 43 related to a particular chemical synthesis may be compared across a plurality of peer chemical synthesizers 1 to determine, for example, whether the chemical synthesis is being reproducibly performed.

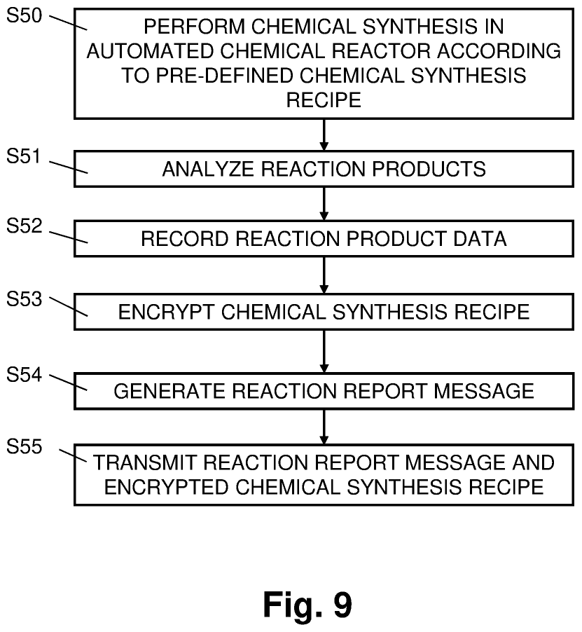

FIG. 9 shows a flow diagram illustrating a number of exemplary steps for providing, by a source chemical synthesizer 2, a chemical synthesis recipe, in particular for use by a peer chemical synthesizer 1. The source chemical synthesizer 2 as described herein includes, by default, the same components as the peer chemical synthesizer 1, in particular the same automated chemical reactor 12. The source chemical synthesizer 1 additionally typically requires the analytics module 14.

In a step S50, a chemical synthesis is performed in the automated chemical reactor 12 in a source chemical synthesizer 2 according to a pre-defined chemical synthesis recipe.

The chemical synthesis is controlled by the control module 11. The pre-defined chemical synthesis recipe may be defined manually by a user, for example using user input, or defined automatically on the basis of a chemical fingerprint which identifies a chemical synthesis.

In a step S51, reaction products of the chemical synthesis are analyzed by the analytics module 14 and reaction product data generated therefrom, analogously to step S41 described herein with reference to FIG. 8.

In a step S52, the reaction product data is received and recorded by the control module 11, analogously to step S42 described herein with reference to FIG. 8.

In a step S53, the chemical synthesis recipe is encrypted in the control module 11, using a cryptographic key.

The cryptographic key used by the source chemical synthesizer 2 for encrypting the chemical synthesis recipe may be a cryptographic key which corresponds to a particular peer chemical synthesizer 1. The cryptographic key may be, for example, a symmetric key shared between the source chemical synthesizer 2 and the particular peer chemical synthesizer 1. The symmetric key may be established using known techniques such as public key cryptography.

In a step S54, the source chemical synthesizer 2, in particular the control module 11 thereof, generate a reaction report message comprising the reaction product data.

In a step S55, the reaction report message and the encrypted chemical synthesis recipe are transmitted to the blockchain platform 6. The reaction report message and the encrypted chemical synthesis recipe may be transmitted as part of a recipe message 3 generated by the source chemical synthesizer 2.

Thereby, the encrypted chemical synthesis recipe may be retrieved and/or used by the peer chemical synthesizer 1 for performing a chemical synthesis according to the predefined chemical synthesis recipe.

In particular, the reaction report message and the encrypted chemical synthesis recipe may be transmitted in the form of a transaction on the blockchain platform 6. The transaction may be configured to originate from a sender address assigned to an address of the source chemical synthesizer 2 on the blockchain platform 6. The transaction may be addressed to a specific target address, the specific target address may correspond to a smart contract on the blockchain platform 6 or an address of a particular peer chemical synthesizer 1.

1-11. (canceled)

12. A method for performing a chemical synthesis on a chemical synthesizer, the chemical synthesizer comprising an automated chemical reactor, a communication module, and a control module, wherein the control module is connected to the automated chemical reactor and the communication module, and the method comprises:
receiving, in the control module using the communication module, a recipe message from a blockchain platform, the recipe message comprising an encrypted chemical synthesis recipe;
decrypting, in the control module, the encrypted chemical synthesis recipe using a cryptographic key; and
performing, in the automated chemical reactor, the chemical synthesis according to the chemical synthesis recipe.

13. The method according to claim 12, wherein the chemical synthesizer further comprises an input module and the method further comprises:
receiving, in the control module via the input module, an input indicative of a reaction identifier;
transmitting, by the control module using the communication module, to the blockchain platform, a selection message indicative of the reaction identifier; and
receiving, by the control module using the communication module, the recipe message associated with the reaction identifier.

14. The method according to one of claim 12, wherein the method further comprises:
generating, in the control module, a confirmation message, the confirmation message comprising a reaction identifier of the chemical synthesis recipe and a chemical synthesizer identifier, the confirmation message being indicative of an initiated chemical synthesis; and
transmitting, by the control module using the communication module, the confirmation message to the blockchain platform.

15. The method according to one of claim 12, wherein the chemical synthesis recipe comprises indicators of control parameters of a chemical synthesis in a chemical synthesizer.

16. The method according to claim 15, wherein the method further comprises:
logging, by the control module, control parameters of the chemical synthesizer during chemical synthesis, preferably in realtime; and
recording, by the control module, control parameter data generated from the logged control parameters.

17. The method according to claim 14, wherein the chemical synthesizer further comprises an analytics module and the method further comprises:
analyzing, using the analytics module, reaction products of the reaction and generating, in the analytics module, reaction product data based on the analyzed reaction products; and
recording, by the control module, reaction product data from the analytics module.

18. The method according to one of claim 16, wherein the method further comprises:
generating, by the control module, a reaction report message including at least one of: the control parameter data or the reaction product data;
encrypting, by the control module, the reaction report message; and
transmitting, by the control module using the communication module, the report message to the blockchain platform.

19. A method for providing, by a source chemical synthesizer, a chemical synthesis recipe, in particular for use by a peer chemical synthesizer, the method comprising:
performing, in the source chemical synthesizer comprising an automated chemical reactor, an analytics module, a communication module, and a control module, a chemical synthesis in the automated chemical reactor according to a predefined chemical synthesis recipe;
analyzing, in the source chemical synthesizer, reaction products of the chemical synthesis using the analytics module and generating, in the analytics module, reaction product data based on the analyzed reaction products;
receiving and recording, in the source chemical synthesizer, reaction product data from the analytics module;
encrypting, in the control module, the chemical synthesis recipe using a cryptographic key;
generating, in the control module, a reaction report message comprising the reaction product data; and
transmitting, using the communication module, the reaction report message and the encrypted chemical synthesis recipe to a blockchain platform, in particular for use by the peer chemical synthesizer.

20. A chemical synthesizer comprising an automated chemical reactor, a communication module and a control module, wherein the control module is connected to the automated chemical reactor and the communication module, and the control module is configured to:
receive, using the communication module, a recipe message from a blockchain platform, the recipe message comprising an encrypted chemical synthesis recipe;
decrypt the encrypted chemical synthesis recipe using a cryptographic key securely stored in the control module; and
perform, using the automated chemical reactor, the chemical synthesis according to the chemical synthesis recipe.

21. The chemical synthesizer according to claim 20, the chemical synthesizer further comprises an input module and the control module is further configured to:
receive, via the input module, an input indicative of a reaction identifier;
transmit, using the communication module, to the blockchain platform, a selection message indicative of the reaction identifier; and
receive, using the communication module, the recipe message associated with the reaction identifier.

22. The chemical synthesizer according to claim 20, wherein the control module is further configured to:
generate, a confirmation message, the confirmation message comprising a reaction identifier of the chemical synthesis recipe and a chemical synthesizer identifier, the confirmation message being indicative of an initiated chemical synthesis; and
transmit using the communication module, the confirmation message to the blockchain platform.

23. The chemical synthesizer according to claim 20, wherein the chemical synthesis recipe comprises indicators of control parameters of a chemical synthesis in a chemical synthesizer.

24. The chemical synthesizer according to claim 23, wherein the control module is further configured to:
log control parameters of the chemical synthesizer during chemical synthesis, preferably in realtime; and
record control parameter data generated from the logged control parameters.

25. The chemical synthesizer according to claim 22, wherein the chemical synthesizer further comprises an analytics module,
wherein the analytics module is configured to analyze reaction products of the reaction and generate reaction product data based on the analyzed reaction products, and
wherein the control module is further configured to record reaction product data from the analytics module.

26. The chemical synthesizer according to claim 24, wherein the control module is further configured to:
generate a reaction report message including at least one of: the control parameter data or the reaction product data;
encrypt the reaction report message; and
transmit, using the communication module, the report message to the blockchain platform.
